(19) United States
(12) Patent Application Publication
(10) Pub. No.: US 2006/0052854 A1
(43) Pub. Date: Mar. 9, 2006

(54) Cerebral temperature control

(76) Inventors: Mats Allers, Lund (SE); Anders Lunderquist, Lund (SE); Fredrik Boris-Moller, Lund (SE); Tadeusz Wieloch, Lund (SE)

Correspondence Address: ALBIHNS STOCKHOLM AB, BOX 5581, LINNEGATAN 2, SE-114 85 STOCKHOLM; SWEDENn, STOCKHOLM, SE

(21) Appl. No.: 10/502,574

(22) PCT Filed: Feb. 5, 2003
(86) PCT No.: PCT/SE03/00187

§ 371 Date: Jul. 28, 2004

(30) Foreign Application Priority Data
Jun. 5, 2000 (SE) 0002100-6

(51) Int. Cl. A61F 7/00; A61F 7/12

(52) U.S. Cl. 607/105; 607/113

(57) ABSTRACT

The present invention relates to a method and equipment for controlling the temperature of the brain or brain hemisphere. More specifically the present invention refers to a method and equipment for quick and efficient control of the temperature of the brain and for maintaining a low temperature in the brain. The inventive concept can be divided into three phases. Firstly, an emergency whole body temperature control phase providing a quick temperature control at for example a scene of accident. Secondly, a brain-selective temperature control phase providing a more efficient temperature control at for example an emergency room and thirdly, a maintained temperature control phase providing an even more efficient temperature control for a selected period of time.

### TECHNICAL FIELD

The present invention relates to a method and equipment for controlling the temperature of a brain of a living being.

### BACKGROUND OF THE INVENTION

In pathological conditions, the body temperature or the temperature of the body parts of a living being influences the healing process and the risk of permanent damage. Cancer cells, for example, are heat sensitive and a local heating of the blood flow around a cancer tumor may for some types of cancer constitute a treatment resulting in restrained tumor growth, or in some cases even in a shrinking of the tumor. In other cases cooling of a body part may be important to reduce adverse secondary symptoms of the pathological condition.

In the case of a stroke, the blood flow in the brain is reduced due to a hemorrhage or the clogging of a blood vessel. This condition is critical and it is important that treatment is initiated at an early stage, to reduce the loss of bodily functions, such as paralysis. It is well known that cooling the brain effectively blocks the development of cellular damage after an episode of ischemia. Cooling of the patient therefore also results in a reduction of the symptoms of neurological deficit. However, there are certain problems associated with the cooling of an entire patient. One is that the cooling takes a considerable amount of time, another that it must be carried out under close control of vital signs or under anesthesia, and a third that there is a risk of cardiovascular complications.

In the case of a circulatory arrest, the brain can suffer permanent damage if the arrest exceeds a time period of about 5-15 minutes. However, if the temperature of the brain is lowered before or after the arrest the brain damage is diminished. This may also be applied to the situation of neonatal asphyxia.

In the case of brain trauma the brain suffers from open or close head concussion. Hypothermia has been shown to diminish traumatic brain injury in such cases.

There are several methods in the prior art to carry out a more isolated cooling of a single organ or body part. An example of cooling of the brain in a human being is disclosed in the patent document WO 98/23917, relating to a method of cerebral retro-perfusion and retro-infusion, involving the cooling of arterial blood that then is returned to the entire brain. However, this method entails a large and complicated surgical procedure, which delays the onset of an actual treatment.

The U.S. Pat. No. 5,906,588 discloses a method and a device for heart-lung bypass and cooling of a specific body part. This disclosure primarily relates to complicated heart surgery and organ transplantation.

Today there is no safe and simple method disclosed for a rapid induction of brain hypothermia in ischemic disease.

### PURPOSE OF THE INVENTION

A purpose of the present invention is to provide a system and a method for quick and efficient control of the temperature of the brain and a route for fast delivery of drugs to the brain. An aim of the invention is to provide a system and a method that is simple and initially does not require specialized personnel acquainted with for example radiology or other diagnostic imaging techniques.

Another purpose of the invention is to provide a system and a method for quick and simple temperature control that also supports subsequent diagnostic measures such as magnetic resonance imaging (MRI), i.e. temperature control without inserting metal components into the body of the patient.

Yet another purpose of the invention is to provide a system and a method for maintaining the temperature control of the brain.

### BRIEF DESCRIPTION OF THE INVENTION

According to the invention the purpose indicated above is achieved by in a first phase, cf. FIG. 1a, introducing an infusion catheter for infusion of a temperature controlled infusion solution or perfusate into a vein initiating a quick general body hypothermia.

In an optional second phase of the invention a second infusion catheter is introduced into an artery of the living being. The second infusion catheter is configured to provide selective temperature control of the brain and infusion of other important substrates and pharmacological compounds into the brain. This accomplishes a quick temperature change in the brain, involving only a comparatively minor procedure.

When treating for example a case of stroke, the brain and thus also the affected brain hemisphere can be cooled quickly according to the method of the invention, resulting in a reduction of the symptoms of functional loss. Preferably, continued cooling is maintained in order to keep the temperature of the brain lowered for as long as it takes to diagnose, medicate and restore the functions of the ischemic brain section.

A further embodiment and possibly a third phase of the invention involves cooling or heating, of blood withdrawn from a living being before the blood is recycled to the living being. In this embodiment an extra-corporeal circuit or conduit is established. In one embodiment the extra-corporeal circuit is established between a vein, e.g. a vein in the lower part of the body, and an artery, e.g. the arteria carotis communis, sinister or dexter, arteria subclavia, brachiocephalic trunk, or some other artery, e.g. an artery that supplies blood to the brain, wherein the temperature of the blood is modified outside the body before the blood is returned to the body through the artery.

Another embodiment contains the method to reintroduce heated blood to the venous system in order to avoid whole body hypothermia. This embodiment thus allows for heating a first part of the body at the same time as a second part, for example the brain, is cooled. The system for performing this embodiment preferably comprises two separate flow branches or circuits, one for the cooling and one for the heating.

Yet another embodiment of the invention involves, in addition to cooling or heating the blood, controlling the oxygenation of the brain and the affected brain hemisphere, i.e. the blood is oxygenated or deoxygenated before it is returned to the body.

The invention comprises a system, equipment components and a method in inter alia the following aspects of the invention:

A method for brain hypothermia, said method comprising, in a first phase to enable an early and fast onset of the hypothermia, the steps of:
providing a container with an infusion solution having a first temperature and a venous infusion catheter connected to an outlet of said container, said venous infusion catheter having an infusion solution lumen;
percutaneously inserting a distal end of said venous infusion catheter into a peripheral vein;
cooling the infusion solution to a second temperature lower than said first temperature; and
infusing a first amount of said cold infusion solution into said vein via the infusion solution lumen of said venous infusion catheter shortly after said cooling, to enable the cold infusion solution to cool the blood flowing to the brain while avoiding air bubbles arising in the infusion solution.

Varieties of this method further comprises a second hypothermia phase for brain-selective hypothermia, wherein an arterial infusion catheter is inserted into an artery and a second amount of cold solution is infused into the arterial system, to enable a more efficient temperature regulation of the brain.

The arterial infusion catheter is inserted into a selected peripheral artery, e.g. an arteria radialis, arteria ulnaris or an arteria brachialis. The method may further comprise the step of positioning a distal tip of said arterial infusion catheter in a selected central artery at the vicinity of a branch artery supplying blood to the brain. The selected central artery is e.g. arteria subclavia at the vicinity of arteria carotis, truncus brachiocephalicus or ascending aorta. In some cases it may be necessary or improving to apply a pressure from the outside of the extremity with the peripheral artery for decreasing peripheral blood circulation.

Further embodiments comprise the steps of:
percutaneously inserting a temperature sensor in a blood vessel draining blood from the brain;
sensing the temperature in the blood of said blood vessel thus providing an indication of the temperature in the brain or selected parts of the brain;
adjusting the infusion rate dependent on said sensed temperature for achieving a desired temperature in the brain.

In one embodiment the emergency phase may be directly followed by a hypothermia phase for maintained hypothermia, comprising the steps of:
inserting into a blood vessel an extraction catheter for extraction of blood;
inserting an arterial infusion catheter in or in the vicinity of an artery supplying blood to the brain;
establishing an first extra-corporeal blood circuit for cooled blood between said extraction catheter and said arterial infusion catheter via a pumping means and a temperature regulating device capable of cooling extracted blood;
extracting blood from said blood vessel via said extraction catheter leading a first amount of said extracted blood into said first extra-corporeal blood circuit;
cooling said first amount of said extracted blood;
infusing said cooled extracted blood to said brain supplying artery via said arterial infusion catheter;
maintaining cooling and circulation in said first extra-corporeal blood circuit for a selected period of time.

A further developed embodiment comprises the steps of:
inserting a venous infusion catheter into a vein of the venous system;
establishing a second extra-corporeal blood circuit for heated blood between said extraction catheter and said venous infusion catheter via said pumping means and a heating device capable of heating extracted blood;
leading a second amount of said extracted blood from said blood vessel via said extraction catheter into said second extra-corporeal blood circuit;
heating said second amount of said extracted blood;
infusing said heated second amount of extracted blood to said venous system via said venous infusion catheter;
maintaining heating and circulation in said second extra-corporeal blood circuit for a selected period of time.

For regulating the temperature this method embodiment further comprises the steps of:
percutaneously inserting a temperature sensor in a blood vessel draining blood from the brain;
sensing the temperature in the blood of said blood vessel thus providing an indication of the temperature in the brain or in a part of it;
adjusting the infusion rate of said cooled blood dependent on said sensed temperature for achieving a desired temperature in the brain, or the steps of:
percutaneously inserting a temperature sensor in a blood vessel draining blood from the brain;
sensing the temperature in the blood of said blood vessel thus providing an indication of the temperature in the brain;
adjusting the temperature of said cooled blood dependent on said sensed temperature for achieving a desired temperature in the brain.

One embodiment comprises a third hypothermia phase for maintained hypothermia or follows the brain-selective hypothermia phase, the third hypothermia phase comprising the steps of:
inserting into a blood vessel an extraction catheter for extraction of blood;
inserting the tip of an optional arterial infusion catheter in or in the vicinity of an artery supplying blood to the brain;
establishing a first extra-corporeal blood circuit for cooled blood between said extraction catheter and said arterial infusion catheter via a pumping means and a temperature regulating device capable of cooling extracted blood;
extracting blood from said blood vessel via said extraction catheter leading a first amount of said extracted blood into said first extra-corporeal blood circuit;
cooling said first amount of said extracted blood;
infusing said cooled extracted blood to said brain supplying artery via said arterial infusion catheter;
maintaining cooling and circulation in said first extra-corporeal blood circuit for a selected period of time, and possibly also the steps of:
inserting a venous infusion catheter into a vein of the venous system;
establishing a second extra-corporeal blood circuit for heated blood between said extraction catheter and said venous infusion catheter via said pumping means and a heating device capable of heating extracted blood;
leading a second amount of said extracted blood from said blood vessel via said extraction catheter into said second extra-corporeal blood circuit;
heating said second amount of said extracted blood;
infusing said heated second amount of extracted blood to said venous system via said venous infusion catheter;
maintaining heating and circulation in said second extra-corporeal blood circuit for a selected period of time.

In its most basic form an embodiment of the emergency phase method comprises the steps of:
providing a container with a cold infusion solution and an infusion catheter connected to an outlet of said container, said infusion catheter having an infusion solution lumen;
percutaneously inserting a distal end of said infusion catheter into a blood vessel;
infusing the cold infusion solution into said blood vessel, to enable the cold infusion solution to flow distally to the brain;
or alternatively phrased, infusing a solution having a first predetermined temperature into a blood vessel until the body has reached a predetermined temperature or a predetermined maximum amount of solution has been infused.

An embodiment of an equipment for brain hypothermia in a living being, comprises:
a container of infusion solution;
a temperature regulating apparatus for said infusion solution;
a flexible elongated infusion catheter, said catheter having a proximal end being attachable to an outlet of said container, said catheter having a sufficiently small diameter to be percutaneously insertable into a blood vessel and its tip forwarded to or into the vicinity of the blood vessel feeding the brain with blood so that the infusion solution will find its way to the brain.

Other aspects of the invention include:

An equipment for brain hypothermia, said equipment comprising, to enable an early and fast onset of the hypothermia:
a container with an infusion solution having a first temperature and a venous infusion catheter being connectable to an outlet of said container, said venous infusion catheter having an infusion solution lumen;
said venous infusion catheter having a distal end devised to be percutaneously inserted into a peripheral vein;
a cooling device being configured for cooling the infusion solution to a second temperature lower than said first temperature;
wherein the cooling device is configured for cooling the infusion solution to a second temperature in the range of 0-37 degrees Celsius;
wherein the cooling device is configured for cooling the infusion solution to a second temperature in the range of 0-4 degrees Celsius;
wherein the infusion catheter is configured to be inserted into a median cubital vein:
wherein the infusion catheter is configured to be inserted into a saphenous vein;
wherein the infusion solution is a hypotonic saline solution;
wherein said first amount of infusion solution is in the range of 1-2 liters or in the range of 10-60 ml/kg;
wherein the infusion solution has an altered osmolarity, e.g. a low osmolarity, in order to lessen the circulatory volume load of the infusion solution when infused into the patient;
wherein the infusion solution is provided in a container that is air-sealed at steady state at a temperature in the range of 37 degrees Celsius;
the equipment further comprising a container with gas or a gas mixture having brain protective properties and equipment for inhaling a controlled fraction of said gas.

An embodiment for equipment configured use in a brain-selective hypothermia phase comprises an arterial infusion catheter configured to be inserted into an artery and a container with a second amount of cold solution configured to be infused into the arterial system, to enable a more efficient temperature regulation of the brain in a second hypothermia phase for brain-selective hypothermia.

In different embodiments this equipment:
the arterial infusion catheter is configured to be inserted into a selected peripheral artery;
wherein the arterial infusion catheter is configured to be inserted into an arteria radialis; arteria ulnaris or an arteria brachialis;
wherein said arterial infusion catheter further is configured to the positioning of a distal tip of said arterial infusion catheter in a selected central artery at the vicinity of a branch artery supplying blood to the brain, wherein said selected central artery is arteria subclavia at the vicinity of arteria carotis, truncus brachiocephalius or ascending aorta.

Embodiments may further comprise a device for applying a pressure from the outside of the extremity with the peripheral artery for decreasing peripheral blood circulation. Further embodiments further comprises:
a temperature sensor configured to be percutaneously inserted in a blood vessel draining blood from the brain; and being configured to:
sensing the temperature in the blood of said blood vessel thus providing an indication of the temperature in the brain; and
adjusting the infusion rate dependent on said sensed temperature for achieving a desired temperature in the brain.

A further developed embodiment is followed by a third hypothermia phase for maintained hypothermia, and comprises:
an extraction catheter being configured to be inserted into a blood vessel for extraction of blood;
an arterial infusion catheter being configured to be inserted into the vicinity of an artery supplying blood to the brain:
coupling means for establishing a first extra-corporeal blood circuit for cooled blood between said extraction catheter and said arterial infusion catheter via a pumping means and a temperature regulating device capable of cooling extracted blood; and being configured to:
extracting blood from said blood vessel via said extraction catheter leading a first amount of said extracted blood into said first extra-corporeal blood circuit;
cooling said first amount of said extracted blood;
infusing said cooled extracted blood to said brain supplying artery via said arterial infusion catheter;
maintaining cooling and circulation in said first extra-corporeal blood circuit for a selected period of time; and possibly further comprising:
a venous infusion catheter being configured to be inserted into a vein of the venous system; and further being configured to:
establishing a second extra-corporeal blood circuit for heated blood between said extraction catheter and said venous infusion catheter via said pumping means and a heating device capable of heating extracted blood;
leading a second amount of said extracted blood from said blood vessel via said extraction catheter into said second extra-corporeal blood circuit;
heating said second amount of said extracted blood;
infusing said heated second amount of extracted blood to said venous system via said venous infusion catheter;
maintaining heating and circulation in said second extra-corporeal blood circuit for a selected period of time.

In this embodiment a temperature sensor would be configured to adjusting the infusion rate of said cooled blood dependent on said sensed temperature for achieving a desired temperature in the brain; or to adjusting the temperature of said cooled blood dependent on said sensed temperature for achieving a desired temperature in the brain.

An embodiment of an equipment for brain hypothermia comprises, to enable a brain-selective hypothermia:
a container with an infusion solution having a first temperature and an arterial infusion catheter connectable to an outlet of said container, said arterial infusion catheter having an infusion solution lumen;
a distal end of said arterial infusion catheter being configured to be percutaneously inserted into an artery in or in the vicinity of a branch artery supplying blood to the brain;
a cooling device configured to cooling the infusion solution to a second temperature lower than said first temperature, to enable the cold infusion solution to cool the blood flowing to the brain while avoiding air bubbles arising, in the infusion solution and an efficient temperature regulation of the brain.

An embodiment of equipment for brain hypothermia comprises, to enable a maintained hypothermia:
an extraction catheter configured to be inserted into a blood vessel for extraction of blood;
an arterial infusion catheter configured to be inserted in an artery into the vicinity of an artery supplying blood to the brain;
means for establishing an second extra-corporeal blood circuit for cooled blood between said extraction catheter and said arterial infusion catheter via a pumping means and a cooling, device capable of cooling, extracted blood;
a venous infusion catheter being, configured to be inserted into a vein of the venous system;
means for establishing a first extra-corporeal blood circuit for heated blood between said extraction catheter and said venous infusion catheter via said pumping, means and a heating device capable of heating extracted blood;
means for extracting blood from said blood vessel via said extraction catheter into said first and second extra-corporeal blood circuit;
a cooling, device for cooling a second amount of said extracted blood;
a heating, device for heating a first amount of said extracted blood; and being configured to:
infusing said cooled second amount of extracted blood to said brain supplying artery via said arterial infusion catheter:
infusing, said heated first amount of extracted blood to said venous system via said venous infusion catheter.

A basic embodiment of an equipment for brain hypothermia, comprises:
a container with a cold infusion solution and an infusion catheter connectable to an outlet of said container, said infusion catheter having, an infusion solution lumen;
a distal end of said infusion catheter being configured to be percutaneously inserted into a blood vessel that supplies the brain with blood;
and being configured to infusing, the cold infusion solution into said blood vessel, to enable the cold infusion solution to flow distally to the brain.
A specifically developed catheter, wherein: the catheter being, configured to assume a curvature at its distal part; comprises
a first lumen having a plurality of openings positioned close to a distal end of the catheter and at the outer arc of the curvature:
a second lumen having an opening, at the tip of the distal end of the catheter;
a distal part of the catheter tapering from said plurality of openings to said tip of the catheter.

### DESCRIPTION OF THE DRAWINGS

The present invention will be described in further detail below, with reference to the accompanying drawings, of which

FIG. 1a is a block diagram indicating three phases for regulating the brain temperature according to embodiments of the invention;

FIG. 1b is a block diagram indicating the steps of a method according to one embodiment of the invention;

FIG. 2 is a schematic illustration of an example of a first embodiment of the system according to the invention, here also called the emergency whole body control phase;

FIG. 3a is a block diagram indicating the steps of a method according to one embodiment of the invention;

FIG. 3b illustrates schematically an example of how a catheter is arranged in the arteria subclavia dexter, in a second embodiment of the system according to the invention, here also called the brain selective temperature control phase;

FIG. 4 is a block diagram indicating the steps of a method according to one embodiment of the invention;

FIG. 5 shows schematically an example of a double lumen catheter applicable in embodiments of the invention;

FIG. 6a illustrates schematically a first example of a the inventive system configured in an embodiment here called the maintained temperature control phase;

FIG. 6b illustrates schematically a second embodiment of the inventive system configured in an embodiment of the maintained temperature control phase;

FIG. 6c illustrates schematically a third embodiment of the inventive system configured in an embodiment of the maintained temperature control phase;

FIG. 7a, 7b and 7c show schematically embodiments of a temperature regulating device;

FIG. 8 illustrates schematically an embodiment of an extra-corporeal blood circuit comprising a shunt; and

FIG. 9 shows schematically an example of a triple lumen catheter applicable in embodiments of the invention.

### DETAILED DESCRIPTION OF THE INVENTION

The present invention relates to a method, a system and a set of disposable equipment components for controlling the temperature of the brain of a living being. More specifically the present invention refers to a method, a system, and a set of disposable equipment for accomplishing a simple, quick and efficient control of the temperature of a brain. The method according to the invention for accomplishing control of the temperature of the brain and maintaining the control of the temperature during a predetermined period of time can be divided into three phases or procedures. Firstly, an emergency whole body temperature control phase, secondly, a brain selective temperature control phase and thirdly, a maintained temperature control phase.

Below, the invention will be explained by reference to examples of embodiments thereof, primarily relating to cooling the brain in a case of stroke, trauma or neonatal asphyxia, or before, during or after a circulatory arrest in a human patient. In some embodiments of the invention the control of the temperature is performed in conjunction with another treatment procedure, such as infusion or inhalation of pharmaceuticals, a gas or a gas mixture, respectively that have brain protective properties. Examples of such gases are different anaesthetic gases such as Isofluran or hydrogen gas. By substances having brain protective properties it is here understood such substances that:
decreases the effects of the occurrence of free radicals, that is a substance scavenging free radicals;
decreases the activation of leukocytes;
decreases inflammatory reactions;
preventing the detrimental cell signaling;
enhancing DNA repair;
preventing dysfunction of mitochondria, endoplastic reticulum (ER), plasma membrane and lysosome;
inhibiting proteases and lipases; or
normalizing lipid metabolism.

There is also a brain protective effect in adding gas or gas mixture in order to optimize, for example decreasing the partial pressure of oxygen.

As mentioned above, in the case of a living being suffering from a stroke or being in a state of resuscitation from circulatory arrest, it is very important to provide a quick cooling of the brain in order to minimize the brain damages due to the reduced or loss of blood supply to the brain. Thus, it is important to provide a method for simple and quick temperature control of the brain that may be performed by nursing or ambulance personnel at for example the scene of an accident or at a hospital. In any case, it is obvious to a person skilled in the art that the invention can be adapted to different uses within the scope of the independent claims.

The Emergency Whole Body Temperature Control Phase

To achieve a quick thermal regulation of the selected brain hemisphere, the perfusion may initially consist of some perfusion solution, such as a saline solution, Ringer's lactate or Ringer's acetate, containing an antioxidant or a drug, followed in a second stage by thermally regulated blood which is returned to the patient. According to this (second) embodiment, a compression cuff, such as a tourniquet or the like, is placed on the arm in question, i.e. the arm into which the device for returning the blood is inserted, in order to suppress peripheral circulation and to increase the pressure in the blood vessel, making the pressure of the returning blood equal to the pressure exerted by the heart on the blood, which in consequence results in a flow velocity of 10-12 ml/s. The flow velocity may optionally be regulated in such a way as to make the blood in the arteria vertebralis dexter and the arteria carotis dexter perfuse with a spill-over to the arcus aortae, the flow along the aorta curvatura major being laminar. This is desirable in order to allow the maximum share of the spill-over to reach the aorta carotis sinister. The above arrangement will prevent flow backwards toward the valvular section.

As soon as the thermal regulation has started, diagnostic work can be initiated, such as magnetic resonance imaging, or some other form of diagnostic examination, without having to abort the thermal regulation as described above. When the periferal blood has reached the desired temperature, for example 32 degrees Celsius, the thermal regulation described is aborted, and the temperature reached can be maintained using conventional cooling blankets or the like.

A simple and quick initial control of the temperature of the brain and the body is achieved by the emergency temperature control phase comprising in more general terms the step of initiating as soon as possible an intravenous infusion of a solution that has a controlled temperature, into a blood vessel of the living being by for example ambulance personnel or other nursing staff. In this connection it is possible to add a temperature controlled solution to the body of a human being in the amount of about 10-60 ml/kg, e.g. 1 to 3 liters, or even more. The solution is for example a saline solution possibly comprising antioxidant or other pharmaceuticals having anti-ischaemic properties or suppressing inflammatory processes. In conjunction also inhalation of selected gases can be provided, which gases may have brain-protective properties. Examples of gases or gas mixtures could be different anaesthetic gases such as Isofluran or hydrogen gas.

By substances having brain protective properties it is here understood such substances that:
decreases the effects of the occurrence of free radicals, that is a substance scavenging free radicals;
decreases the activation of leukocytes;
decreases inflammatory reactions;
prevents the detrimental cell signaling;
enhances DNA repair;
prevents dysfunction of mitochondria, endoplasmatic reticulum (ER), plasma membrane and lysosome;
inhibits proteases and lipases; or
normalizes lipid metabolism.

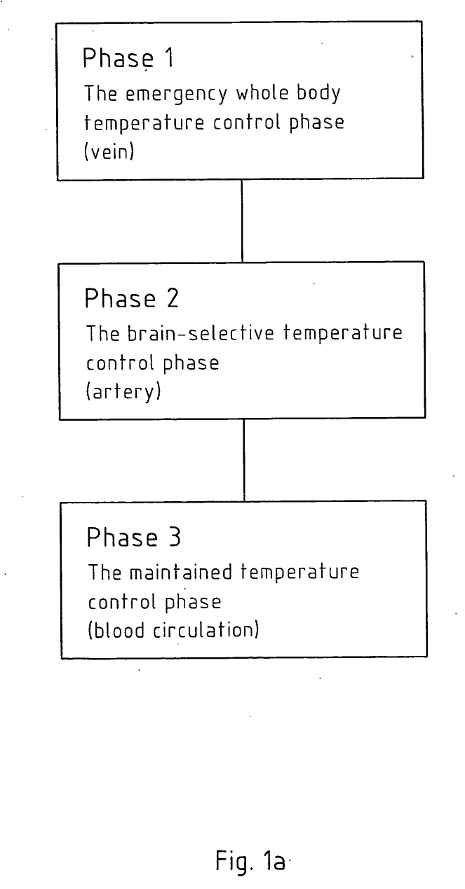

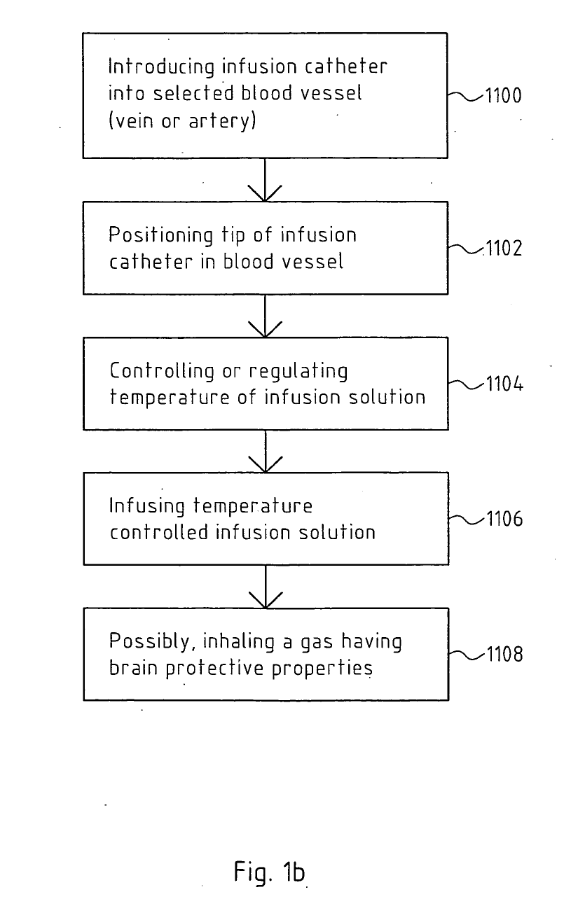

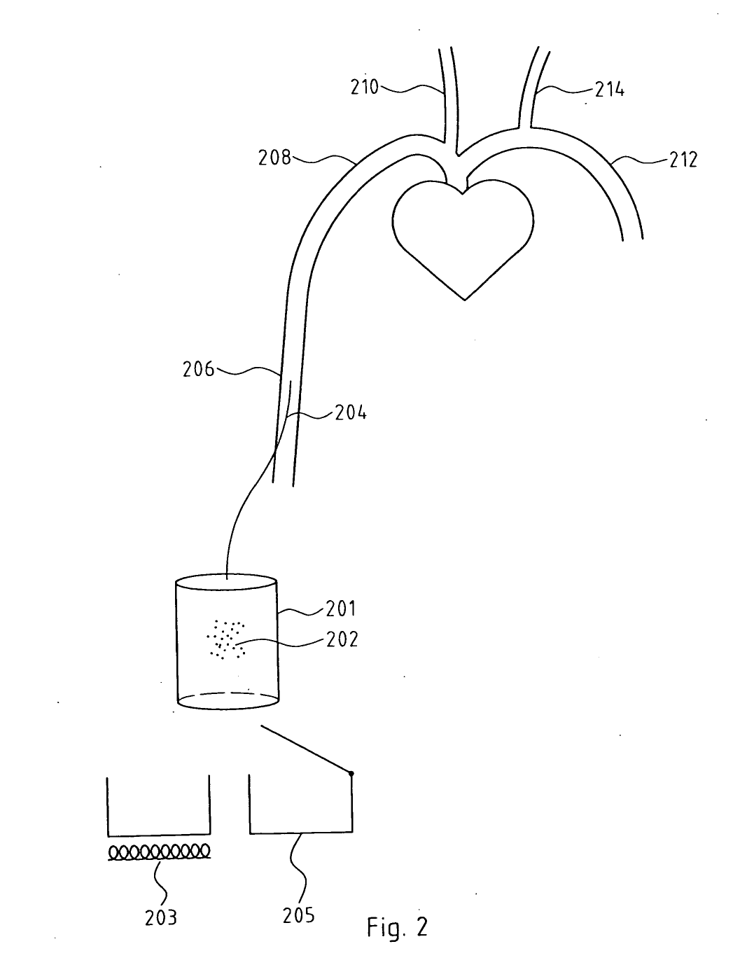

These pharmaceuticals may include calcium blocking substances, magnesium or Nimodipin. However, in this description text referring to the whole body temperature control phase, we will refer to the blood vessel as a vein but it could also be an artery if for example personnel that is competent in arterial catheterization is available. In the case of controlling the temperature during a stroke or a circulatory arrest, the infusion solution is preferably a cold or ice cold solution, e.g., a saline solution possibly comprising an antioxidant. More specifically, embodiments of the emergency temperature control phase comprises as shown in FIG. 1b (cf. FIG. 2) the steps of: 1100 introducing, percutaneously or through a surgical cut down, an infusion catheter into a selected peripheral blood vessel, preferably a vein 206 such as the median cubital vein 206 or saphenous vein, or a central blood vessel 208, 210, 212, 214 such as the jugular vein 210, 214 or the subclavian vein 208, 212; 1102 positioning the tip of the infusion catheter as close to the venous inlet of the heart as possible without getting adverse cardiac effects of the quick infusion of a temperature controlled solution; 1104 controlling or regulating the temperature, e.g. cooling, of an infusion solution by means of a temperature regulating means, e.g. a cooling means; 1106 infusing the temperature controlled infusion solution, e.g., a cold or ice cold saline solution, preferably having an altered osmolarity, e.g. a low osmolarity, and comprising for example ischemic protective pharmaceuticals into the blood vessel.

One embodiment may also possibly comprise the step of 1108 possibly inhaling through a face mask or an endotracheal tube a controlled fraction of gas or a gas mixture with brain-protective properties.

Thus, in step 1100 an infusion catheter is introduced, preferably percutaneously, into a selected blood vessel, preferably a vein, of the living being such as the median cubital vein, saphenous vein, cephalic vein or basilic vein. For simplicity, the chosen venous catheter is designed such and of the type that the involved personnel are well acquainted with. To achieve sufficient flow of the temperature controlled solution, the inner diameter of the infusion catheter is typically in the range of 1-4 mm, preferably in the range of 2-3 mm, and the length of the infusion catheter is at least about 3 cm. In the case of an infusion catheter long enough to reach for example the intrathoracic space, the tip positioning according to step 1102 is preferably performed by means of indication means such as markings arranged on the outer surface of the infusion catheter or small diameter-changes of the catheter, whereby the indication means indicates the length of the catheter that has been introduced into the living being. Thus the position of the catheter tip can be determined without the use of any imaging techniques.

In step 1104 the temperature of the infusion solution is regulated or controlled, i.e. the infusion solution is temperature controlled. In the case of stroke or circulatory arrest the infusion solution is preferably a cooled hypotonic saline solution, i.e. a saline solution having an osmotic pressure lower than the blood. An infusion solution having a low osmolarity is preferably chosen in order to lessen the circulatory volume load of the infusion solution. In for example an ambulance, about one to two liters of infusion solution having a temperature in the range of 0-37 degrees Celsius, preferably 0-10 degrees Celsius, more preferably in the range of 0-4 degrees Celsius, can be infused, whereby a lowered body and brain temperature is achieved. However, in some situations it is sufficient to infuse an infusion solution having a temperature in the range of 10-37 degrees Celsius, since infusion of a infusion solution having a temperature lower than the body temperature will cause a decreased brain temperature.

In one embodiment of the invention, the infusion solution is cooled by means of a peltier element, i.e. an apparatus which by means of electricity, produces cooling of a metal part in an electrical circuit which metal part cools the infusion solution. However, other suitable cooling means can also be used. For example, a container comprising the infusion solution can be placed in an icebox or in a container comprising ice or ice cubes.

However, a cooled infusion solution in steady state with the atmospheric pressure may give rise to gas bubbles or air bubbles of different sizes when supplied to a patient, due to the lower gas solubility at higher temperatures. Such air bubbles may be very harmful to the patient if they are conveyed to the brain. Thus it is important to provide a quick cooling of the solution just before the supply of the solution in order to avoid or decrease the amount of gas or air bubbles in the infusion solution. Another way of avoiding or decreasing the amount of air bubbles is to provide the infusion solution in a sealed or air-sealed container, for example in a sealed container manufactured of a gas impermeable plastic or a plastic-like material, and sealing performed at steady state at a temperature in the range of 37 degrees Celsius.

Further, in step 1106 the infusion solution is infused to venous blood by means of the infusion catheter, whereby a lowered body temperature is achieved. In one embodiment of the invention, the body temperature is decreased by one to two degrees Celsius when one to two liters of cooled infusion solution is infused. The infusion speed or velocity can be varied for example by applying a pressure on the container or plastic bag comprising the infusion solution, but the infusion speed should preferably be as large as possible. However, the infusion speed depends on the dimensions of the infusion catheter.

Possibly, in step 1108 a gas or a gas mixture having brain protective properties is inhaled through for example a facial mask. Examples of such gases are different anaesthetic gases such as NO, Isofluran and hydrogen gas. By substances having brain protective properties it is here understood such substances that:
decreases the effects of the occurrence of free radicals, that is a substance scavenging free radicals;
decreases the activation of leukocytes;
decreases inflammatory reactions;
prevents the detrimental cell signaling;
enhances DNA repair;
prevents dysfunction of mitochondria, endoplasmatic reticulum (ER), plasma membrane and lysosome;
inhibits proteases and lipases; or
normalizes lipid metabolism.

There is also a brain protective effect in adding gas or a gas mixture in order to optimize, for example decreasing, the partial pressure of oxygen and carbondioxide.

FIG. 2 shows this embodiment of the invention applied in a human patient for cooling the brain. A container 201, for example a plastic bag, having a temperature controlled infusion solution 202, is coupled to an infusion catheter 204. The infusion solution is cooled in a cooler 203 before use or is maintained in a cooling box 205. In this exemplifying figure, the infusion catheter is percutaneously introduced into the right median vein 206 and inserted as close to the venous inlet of the heart as possible without getting adverse cardiac effects due to a quick infusion of a cooled infusion solution. The closer the outlet of the infusion catheter 204 is to the venous inlet of the heart the smaller the temperature loss in the cooled infusion solution before reaching the brain. As the emergency temperature control goes on by infusing in this case cooled solution, the temperature of the whole body is decreased.

In the case of heating a cold patient, the treatment would be analogue but of course with a heated infusion solution.

The Brain-Selective Temperature Control Phase

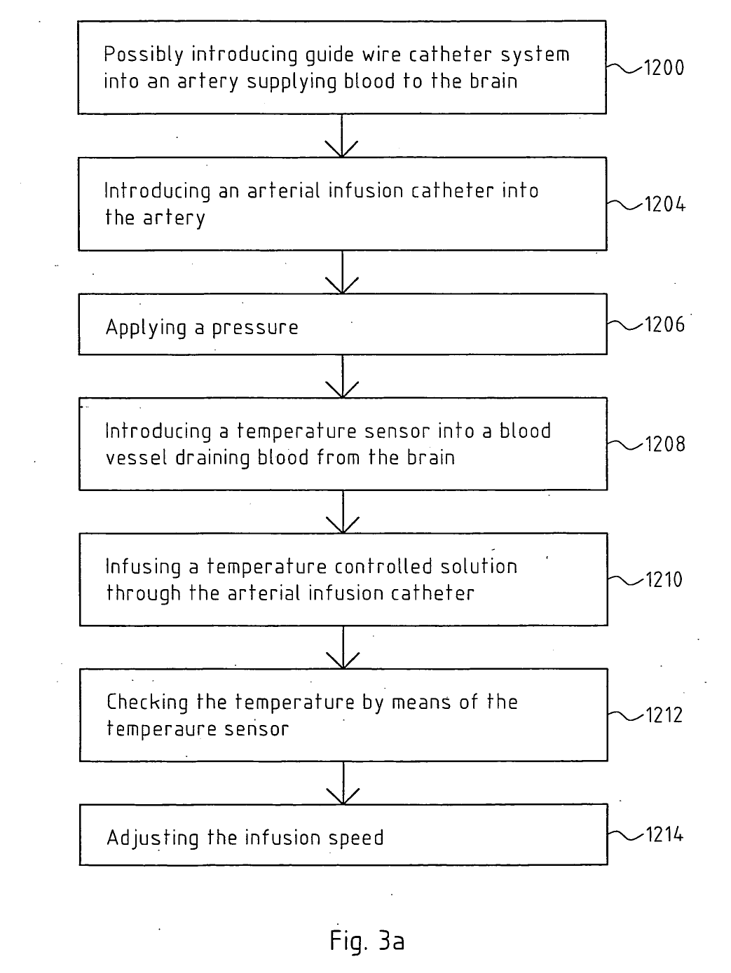

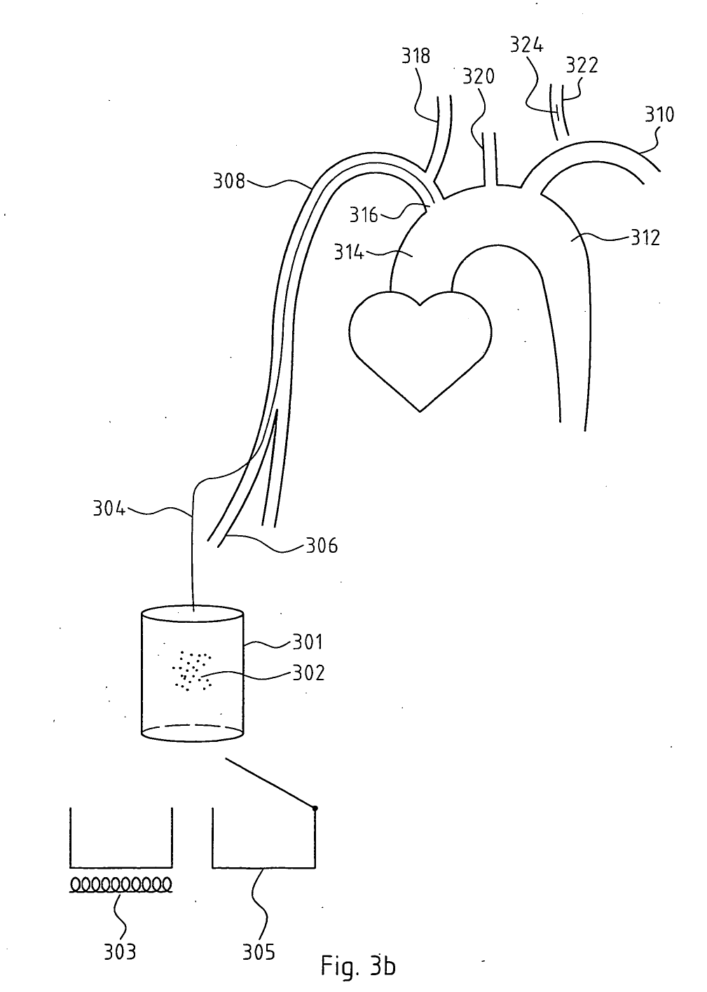

When personnel qualified for arterial catheterization reaches the patient, for example at a hospital, the brain-selective temperature control phase may be achieved, either as a complement to the emergency whole body temperature control procedure or as separate temperature control procedure. As schematically shown in FIG. 3a, the brain-selective temperature control phase comprises the steps of: 1200 possibly introducing by surgical cut-down or percutaneously with e.g. Seldinger technique a guide wire-catheter system into a selected relatively peripheral artery, for example arteria radialis, arteria ulnaris or arteria brachialis to provide a high amount of cooled infusion to the brain; 1202 introducing an arterial infusion catheter into the selected artery, for example arteria radialis or arteria brachialis by means of a guide-wire; 1204 positioning the tip of the arterial infusion catheter in a selected relatively central or brain close artery, preferably in the right arteria subclavia, truncus brachiocephalica, the ascending aorta, arteria carotis communis or in another selected artery; 1206 applying a pressure from the outside on the extremity with the peripheral artery, e.g. the arm or the leg, for example by means of a circumferential torniquet, preventing or decreasing peripheral circulation of the blood, when the cooled solution is infused through the catheter; 1208 preferably introducing percutaneously a temperature sensor, such as a thermistor or a thermocouple, into a blood vessel that drains blood from the brain such as the right and/or the left jugular veins. The temperature sensor is preferably coupled to an externally arranged display means displaying the measured temperature of the blood in the brain draining blood vessel, e.g. the jugular vein(s); 1210 infusing a temperature controlled solution, e.g., a cold or ice cold saline solution, preferably having an altered osmolarity, e.g. a low osmolarity, through the arterial infusion catheter, by means of a supplying means such as a pumping means, e.g., a perfusion pump, to the selected site of infusion; 1212 checking the temperature of the brain or the affected brain hemisphere by means of the temperature sensor sensing the temperature in the blood flow in the respective brain draining blood vessels, e.g. the respective jugular vein, thus being dependent on the brain temperature; 1214 adjusting the infusion speed or the infusion rate, i.e. the amount of the temperature controlled infusion solution supplied per unit of time, so that a desired and predetermined temperature is achieved in the brain or the affected brain hemisphere, thus preferably dependent on the sensed temperature.

Thus in step 1200, a guide wire-catheter system is possibly introduced, percutaneously or by surgical cut-down, into a selected blood vessel supplying blood to the brain. Preferably, the catheter is introduced into the right arteria radialis or the light arteria brachialis, and advanced to the predetermined position, whereby a high concentration of cooled blood can be supplied to the brain. In step 1202, a device for infusion of an infusion fluid, preferably consisting of a guide wire and a heparinized and hydrophilic arterial infusion catheter is inserted. The tip of the arterial infusion catheter is in step 1204 positioned in for example the right arteria subclavia, arteria brachiocephalica or in the ascending aorta.

An external pressure is possibly applied in step 1206 on the arm used for the arterial infusion catheter. The pressure is preferably achieved by means of a pressure means, for example by applying a circumferential tourniquet around the selected arm, the pressure means can further be arranged for cooling the arm or for preventing peripheral circulation of blood.

In step 1208 one or several temperature sensor(-s), such as a thermistor or a thermocouple, is preferably percutaneously introduced into for example the right and/or left jugular veins. The temperature sensor is preferably coupled to an externally arranged display means displaying the measured temperature of the blood in the jugular vein(s) to an operator. It should be understood that measurement of the temperature is preferably performed in the internal jugular vein since this will more accurately reflect the temperature of the brain or brain hemisphere.

In step 1210 a temperature controlled infusion solution, e.g. a cold or ice cold saline solution, is infused into the selected artery by means of the arterial infusion catheter and a supplying means, such as a pumping means, e.g. a perfusion pump.

Further in step 1212, the temperature of the brain or the affected brain hemisphere is controlled by means of the temperature sensor, and in step 1214, the infusion speed of the infusion solution is adjusted to achieve a desired and predetermined temperature in the brain or the affected brain hemisphere.

In one embodiment of the invention, the arterial infusion catheter has a diameter in the range of 0.3-3.3 mm (1-10 F. 1 F=0.33 mm), preferably in the range of 1.3-2.6 mm (4-8 F). The surface of the arterial infusion catheter is preferably coated with an anticoagulant compound to prevent coagulation of blood on the catheter surface and in the surroundings of the arterial infusion catheter. Further, in one embodiment of the invention the arterial infusion catheter has an externally arranged indication means, such as markings or diameter-changes, in order to indicate the position of the catheter tip in the selected artery, e.g. the right arteria subclavia, truncus brachiocephalicus or ascending aorta. Thus it is possible to guide and position the catheter tip into a selected and predetermined position without using any diagnostic imaging method, such as a radiological method. The dimensions and positioning markings are preferably adapted to the size of the body of the patient.

FIG. 3b shows schematically an example of this embodiment of the invention applied in the arterial system of a human patient. A container 301 containing a temperature controlled infusion solution 302 is connected to an arterial infusion catheter 304. The infusion solution 302 is tempered in a cooler or heater 303 or has a pre-arranged temperature that is maintained by means of a cooling or heating box 305. Most examples in the specification are however directed to cooling.

In this figure, the infusion catheter 304 is inserted into the right radial artery 306 with its distal end outlet introduced up the right subclavian artery 308 in a position at level with the inlet of the right carotid artery 318. In this embodiment, a low infusion flow rate will cause the infusion solution to flow up into the right common carotid 318. If the infusion rate is increased a part of the infusion solution will flow up into the right common carotid 318 and another part of the infusion solution will flow down into the ascending aorta 314 and up into the left common carotid 320. However, if the infusion rate is increased even more, the infusion solution will flow down into the coronary arteries, which should be avoided because of possible detrimental effects on the cardiac performance.

In another embodiment of the invention, the infusion catheter 304 is inserted via the right arteria subclavia 308 into the truncus brachiocepahlica 316. In the embodiment, a low infusion rate will cause the infusion solution to flow up into the right common carotid 318. If the infusion rate is increased a part of the infusion solution will flow up into the right common carotid 318 and another part will flow into the ascending aorta 314 and up into the left common carotid 320. However, if the velocity of the infusion solution is increased further, the infusion solution will flow down into the coronary arteries, which should be avoided because of possible detrimental effects on the cardiac performance.

In yet another embodiment of the invention, the infusion catheter 304 is introduced via for example the left arm and the left arteria subclavia 310 into the ascending aorta 314. In this embodiment, the infusion solution will flow up into the left and right common carotids, 320 and 318, respectively.

In a further embodiment of the invention, the infusion catheter 304 is introduced into the ascending aorta 314 via arteria femoralis and the aortic arch 312, whereby the supplied infusion solution flows up in the left and right common carotid, 320 and 318, respectively. This infusion catheter can for example be a double lumen catheter as described below.

Further, a temperature sensor 324 is introduced into a jugular vein 322, whereby the temperature of the blood from the brain hemisphere can be monitored and thus a measure of the temperature in the brain is achieved.

In an embodiment of the invention, the infusion catheter 304 is coupled to a perfusion apparatus that generates a pulsating flow of infusion solution. This pulsating flow is then synchronized to the heart cycle such that the cool infusion solution flows up in the left and right carotids, 320 and 318, respectively, when the heart is in systole, i.e. when the heart is contracting and pumping blood to the arteries.

In an embodiment of the invention, the infused solution has a temperature in the interval of 0-37 degrees Celsius or in the interval of 0-20 degrees Celsius, preferably in the interval of 0-10 degrees Celsius, and most preferably in the interval of 0-4 degrees Celsius. Preferably, the infusion solution is held or stored in room temperature or preferably at 37 degrees Celsius and is quickly cooled just before the start of the infusion procedure to decrease or avoid the risk of formation of gas or air bubbles in the blood vessel when the infusion solution is infused. In one embodiment of the invention, the infusion has an altered osmolarity, e.g. a low osmolarity in order to lessen the circulatory volume load of the infusion solution.

After the insertion of the arterial infusion catheter, one to two liters of infusion solution having a temperature in the range of 0-4 degrees Celsius can be infused intra-arterially without adverse circulatory effects, whereby a selectively lowered brain temperature is achieved as compared to the whole body temperature control procedure. Further, in some cases a brain temperature in the interval of 15 to 37 degrees Celsius is desirable, but in other cases a brain temperature in the range of 30-37 degrees Celsius is desirable. Preferably, a brain temperature in the range of 27 to 35 degrees Celsius is desired and more preferably a brain temperature of approximately 33 degrees Celsius.

According to an embodiment of the invention, a temperature about 30-35 degrees Celsius in the brain or the affected brain hemisphere can be achieved after about 2-8 minutes, by supplying a cooled infusion solution of a temperature in the range of 0-4 degrees Celsius at a speed in the range of 300-700 milliliters per minute. However, in another embodiment a speed in the range of 50-700 milliliters per minute is suitable. The achieved temperature, time and flow rate are probably dependent on body size of the patient as well as on the infusion rate.

The flow velocity of the infusion solution can optionally be regulated in a way making the blood in the arteria vertebralis dexter and the arteria carotis dexter to perfuse with a spill-over to the arcus aortae and to flow along the aorta curvatura major into the left carotic artery. This is desirable in order to allow the maximum share of the spill-over to reach the aorta carotis sinister. The above arrangement will prevent flow backwards towards the valvular section.

The Maintained Temperature Control Phase

The maintained temperature control procedure, i.e., the procedure for maintaining the desired level of selected brain hypothermia or hyperthermia, can either be accomplished during or after the whole body temperature control procedure or the brain-selective temperature control procedure described above. Further, the maintained temperature control phase can be achieved by using for example a temperature regulated blanket or mattress or by establishing an extra-corporeal circulation of temperature regulated blood or another temperature regulated solution. This phase is for example accomplished in hospitals wherein immediate access to specialized personnel in cardiology and radiology is provided.

In the inventive concept, this phase comprises a method of establishing an extra-corporeal circulation of temperature controlled blood. The method comprises: withdrawal or extraction of blood from a living being for example suffering of stroke, trauma, neonatal asphyxia or circulatory arrest, controlling or regulating the temperature of the blood, possibly also regulating the oxygenation of the blood, and reintroducing the blood into the living being. By means of these measures, the temperature of the brain and possibly the rest of the body are controlled. One embodiment of the method comprises the establishment of two extra-corporeal circuits, namely a first circuit for cooling the brain and a second circuit for heating other parts of the body, e.g. for keeping the rest of the body at normal temperature. A variety of this embodiment utilizes a peltier element for cooling and the opposite side of the same peltier element for heating.

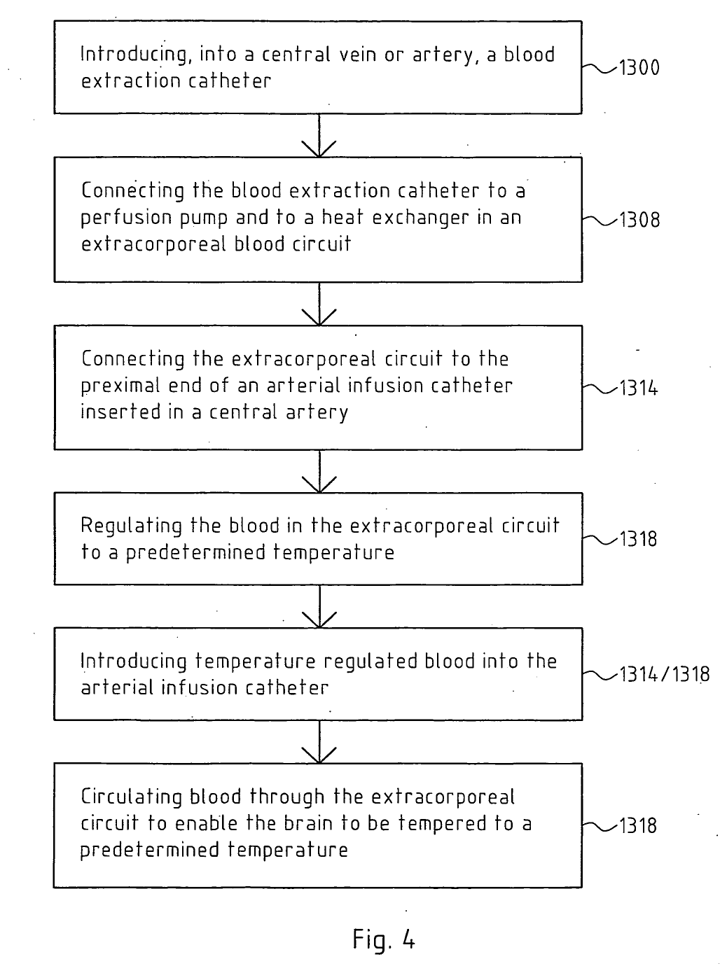

More specifically, an embodiment of the method of establishing an extra-corporeal circulation of temperature controlled blood comprises the steps of (cf. FIG. 4): 1300 introducing, into a central vein or artery of a patient, an extraction catheter for extraction of blood; 1302 possibly, introducing a venous infusion catheter into a vein of the patient; 1304 possibly, introducing an arterial infusion catheter into an artery of the patient, preferably to an artery supplying blood to the brain of the patient; 1306 possibly, introducing one or several temperature sensors into one or both jugular veins, whereby the temperature(-s) of the blood in the jugular vein(-s) can be measured; 1308 connecting the extraction catheter to an arterial cannula, i.e. the arterial infusion catheter, via a perfusion pump, a heat exchanger and possibly also a regulator regulating the oxygenation of the blood, whereby an extra-corporeal circulation to an artery controlling the temperature of the brain is established; 1310 possibly also connecting the extraction catheter to the venous infusion catheter by means of for example a flow-dividing Y-connector, via a second perfusion pump and a heat exchanger, whereby an extra-corporeal circulation to the venous system is established; 1312 circulating venous or arterial blood extra-colporeally from the patient through the oxygenator and heat exchanger(-s), regulating or controlling the oxygenation and temperature of the blood, and back to the patient; 1314 reintroducing temperature controlled blood into the patient through the arterial infusion catheter arranged in an artery leading blood to the brain, whereby the reintroduced blood regulates the oxygenation and temperature of the brain; 1316 optionally, reintroducing temperature controlled blood into the patient through the venous infusion catheter arranged in the vein, whereby the reintroduced blood regulates the temperature of the rest of the body; 1318 maintaining the extra-corporeal circulation regulating the temperature at the desired temperature levels of the brain for a desired period of time thereafter; 1320 possibly, optimizing the viscosity of the blood by infusing a saline solution or by hemofiltration; and 1322 applying a heating or cooling blanket in order to maintain a desired body temperature level over an extended period of time.

In step 1300, a blood extraction means is inserted into a suitable vein or artery, e.g., vena femoralis or arteria femoralis or vena jugularis. The blood extraction device is in one embodiment of the invention an introducer or a first catheter. For example an extraction catheter having an outer diameter smaller than the normal inner diameter of the vein or the artery, so as not to stop entirely the flow of blood around the extraction catheter, but large enough to give a sufficient flow of extracted blood. The vein or artery is e.g. the vena femoralis or arteria femoralis or vena jugularis. For an adult patient an outer diameter of approximately 2.6-4.6 mm (8-14 F) and an inner diameter as large as possible in the relation to the outer diameter would be sufficient. For children or grown-ups with other vein or artery dimensions, the dimensions of the catheter will obviously have to be modified accordingly. The catheter has in one embodiment of the invention, a conical extra-corporeal coupling for low flow resistance during perfusion. This coupling is provided with a seal that can be perforated, for example by a guide wire and/or a dilator, to prevent unnecessary bleeding. The seal can also be removed for attachment to an extra-corporeal circuit. Preferably, the catheter is heparinized internally and externally, to counteract coagulation of the blood that comes into contact with the catheter.

In step 1302, an infusion device is introduced possibly into a sufficiently large peripheral or central vein. The infusion device, is preferably an infusion catheter for the reintroduction or reinfusion of temperature regulated blood or for infusion of a treatment solution. This infusion catheter is primarily intended for the reinfusion of possibly heated extra-corporeally circulated blood, whereby the undesirable cooling effects of the rest of the body may be postponed or completely counteracted.

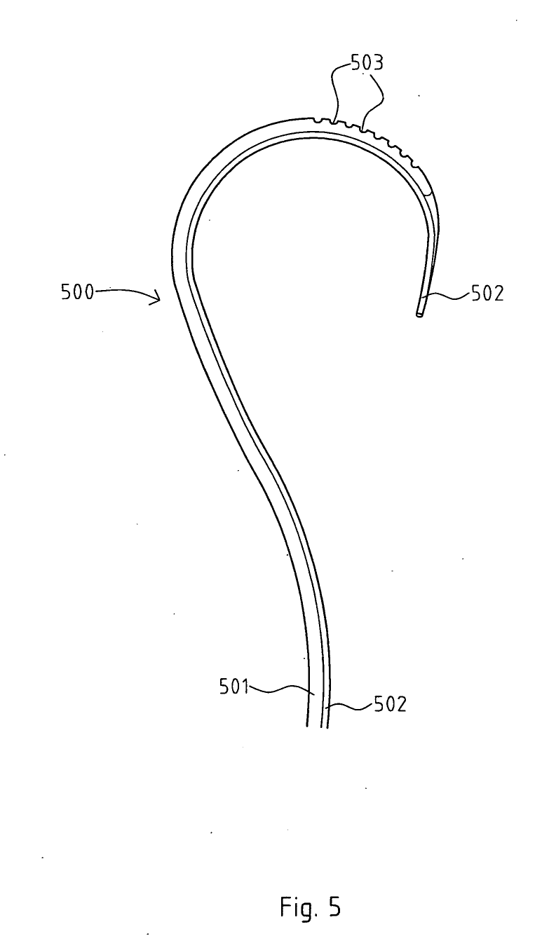

In step 1304, an arterial infusion catheter is possibly introduced in a peripheral artery and its tip positioned into an artery supplying blood to the brain. The arterial infusion catheter can be the one used in for example a previous selective brain temperature control phase, whereby it is not necessary to introduce another arterial infusion catheter. However, another arterial infusion catheter can optionally be introduced, for example a double lumen catheter (cf. FIG. 5), which preferably is introduced into the ascending aorta via a femoralis artery. Thus, the optionally introduced arterial infusion catheter can either be used alone or together with another arterial infusion catheter, for example together with an arterial infusion catheter introduced via the left or right arteria subclavia.

In FIG. 5, an exemplifying embodiment of a double lumen catheter 500 is shown. The double lumen catheter has an outer diameter of about 2.7 mm and a first inner lumen 501 having a diameter of about 2.1 mm. In the wall of the catheter is an extra lumen, viz a second inner lumen 502 having a diameter of about 0.3 mm. The distal end of the catheter tapers over a distance of about 3 cm to 1.3 mm still containing the small second inner lumen. The large first inner lumen ends about 4 cm from the tip. The catheter is shaped as a right coronary artery catheter. Extending from the catheter tip and over a distance of approximately 5 cm are a number, for example eight, side holes 503 to the greater lumen of the catheter arranged along the greater curvature of the catheter. The side holes have a diameter of about 1.5 mm.

When the catheter is in position, the tip of the catheter 500 is positioned slightly above the entrance of the left coronary artery and the side holes to the greater lumen are arranged against the right brachiocephalic artery and left carotid artery. By infusion of an infusion solution, such as temperature controlled or regulated blood or a temperature controlled solution at high infusion rate (5-10 ml per second) by means of the larger first inner lumen 501, the infusion solution will flow through the holes 503 against the right brachiocephalic and left carotid artery, whereby rather selective cooling of the brain can be achieved for inducing or maintaining temperature control.

If necessary, the catheter 500 can be advanced some further centimeters, whereby the tip enters the left coronary artery and pharmacological compounds or radiological contrast may be administered locally to the heart by means of the second inner lumen 502.

At least one temperature sensor or a thermistor is in step 1306 inserted preferably percutaneously into a jugular vein, whereby the temperature of the blood in the vein can be measured. However, in one embodiment of the invention two temperature sensors are arranged in vena jugularis, sinister and dexter, respectively. By means of the temperature sensors good temperature regulation of the brain is supported. Through the vena jugularis interna, the blood is transported away from the brain, and the thermistor gives off a signal that reflects the temperature of the blood leaving the brain, thus indicating the temperature of the brain or brain hemisphere being thermally regulated. The thermistor preferably consists of some suitable disposable material, which preferably is heparinized. The dimensions of the part inserted into the vena jugularis interna should be small enough to prevent any significant obstruction of the venous blood flow.

The output of the thermistor may be used in different ways. In one embodiment, the signal is transferred to a regulator, which controls the heat exchanger or the circulation pump or both, in order to achieve a regulated temperature level in the selected brain hemisphere. Alternatively, or in addition, the signal may be transferred to an indicating device, such as a visual display showing the current temperature of the blood in the vena jugularis interna, and hence the approximate temperature of the brain or the selected brain hemisphere.

In step 1308, the extraction catheter is connected to the arterial cannula, i.e. the return device or the arterial infusion catheter, via a perfusion pump, a heat exchanger and possibly also a regulator regulating the oxygenation of the blood, thereby establishing an extra-corporeal circulation circuit for temperature regulated blood.

Thus the inlet of a blood conduit such as a blood tube is attached according to prior art to the other opening of the extraction catheter, and the tube is passed through a perfusion pump. The blood tube preferably consists of an internally heparinized biocompatible plastic material, and has a diameter suited to its purpose. The blood tube passes through a circulation pump according to prior art, a so-called perfusion pump, preferably equipped with rollers exerting a peristaltic effect externally on the tube.

The blood tube extends from the pump to a heat exchanger, which in this particular embodiment is arranged for cooling the blood, but which in another embodiment may be arranged for heating it or it can be arranged for both heating and cooling. In one type of heat exchanger the blood tube passes through a device which supplies or removes heat energy from the blood through the walls of the blood tube. In another type of heat exchanger, the blood tube is attached to a heparinized heat-exchanging bag with blood canals, providing a large surface area for heating/cooling.

In the embodiment intended for the treatment of stroke or circulatory arrest, the heat exchanger should be capable of regulating blood to a temperature between 0 and 37 degrees Celsius. In some cases, a small temperature fall of only a few degrees is desirable, for example a cooling to 34 degrees Celsius. In other cases a larger temperature fall is desirable, such as down to 0-5 degrees Celsius. Within other areas of application, a heating of the blood may be desirable, such as from 37 degrees Celsius to about 40 degrees Celsius. As the brain or selected brain hemisphere is cooled/heated, the general body temperature also falls/rises, and accordingly the temperature of the extracted blood. The heat exchanger is therefore controlled so as to keep the blood returned to the body after cooling/heating at the desired temperature.

Optionally, the blood conduit may be attached to a per se known oxygenator or a deoxygenator, before or after the heat exchanger, in order to oxygenate or to deoxygenate the blood.

One outlet end of the extra-corporeal blood circuit is attached to the proximal end of the arterial infusion catheter reaching into the right arteria subclavia, truncus brachiocephalicus, ascending aorta or arteria carotis communis, from the heat exchanger or in relevant cases from the oxygenator or deoxygenator, which completes the configuration of the temperature controlling system and makes it ready for use.

In an embodiment of the present invention, the circulation pump is placed in the proximity of the place of extraction of blood, but it can also be placed elsewhere in the extra-corporeal blood circuit, for example immediately before the blood return catheter. In such a case, the rest of the blood conduit should be primed before starting the infusion of the infusion solution.

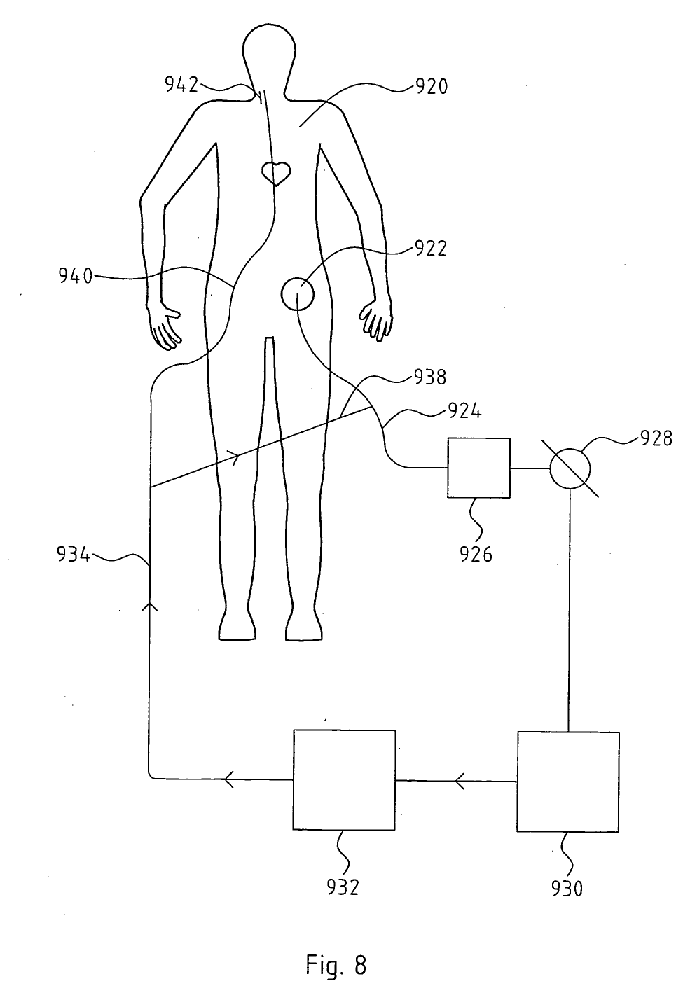

In one embodiment of the invention (cf. FIG. 8), an open reservoir 926 containing, for example, priming solution, such as a saline solution or blood, is arranged between the extraction catheter 922,924 and the circulation pump or the perfusion pump 928, and a shunt 938, in the form of an internally heparinized blood tube, has been arranged extra-corporeally to create a connection from one section between the extraction catheter 922,924 and the reservoir 926 to another section between the artery catheter/infusion catheter 934, 940 and the heat exchanger 932 or the oxygenator/deoxygenator 930. By closing the flow of blood via the extraction catheter 922 and opening the flow from the artery catheter 940, blood will flow out of the arterial catheter 934, and will be pumped by the circulation pump 928 to the reservoir 926, whereby the system will be purged of any air present. Any air present at the extracted blood side, i.e. the venous side or the arterial side, can then be removed similarly by stopping the flow of blood to/from the artery and using the circulation pump 928 to make the extracted blood and air flow to the reservoir 926. When the system has been purged of air, the blood flow through the shunt 938 is stopped, for example by means of an artery forceps, and the circulation of blood can be started and the temperature of the brain can be monitored by means of a temperature sensor 942 arranged in a jugular vein of the patient 920.

As the brain is continuously selectively cooled, the general body temperature also falls, possibly to undesirable levels. To counteract this effect of brain cooling, optionally a part of the reinfusable blood is in step 310 deviated by way of a Y-formed connector into a heating device and a circulation pump and further introduced into the venous system of the patient. The outlet end of this part of the extra-corporeal blood circuit is attached to the proximal end of the venous infusion catheter.

In step 1312, venous or arterial blood is circulated extra-corporeally from the patient through the heat exchanger regulating the temperature of the blood and back to the patient. Thus, when the system for temperature control has been configured, circulation of blood through the extra-corporeal circuit is started, involving the extraction of blood from the vein or artery, cooling or heating it to the desired temperature in the heat exchanger(-s), for example to a temperature between 0 and 37 degrees Celsius, optionally regulating oxygenation by means of an oxygenator, and finally reintroducing it into the patient via the arterial infusion catheter. The temperature controlled blood flows from there into the brain or the affected brain hemisphere, which temperature is controlled swiftly and efficiently.

In step 1314, the temperature controlled blood is reintroduced into the patient through the arterial infusion catheter arranged in an artery leading blood to the brain, whereby the reintroduced blood regulates the oxygenation and temperature of the brain or the selected brain hemisphere.

In step 1316, heated blood, having a temperature in the range of 37-40 degrees Celsius, is optionally reintroduced into the patient through the venous infusion catheter, whereby undesired general cooling effects on the rest of the body are diminished.

In step 1318, the temperature regulated extra-corporeal circulation is maintained at the desired temperature levels of the brain or the affected brain hemisphere for a desired period of time thereafter.

In step 1320, the viscosity of the blood can possibly be optimized by infusing a saline solution diluting the blood or by hemofiltration concentrating the blood.

In step 1322, external cooling by the means of a cooling blanket may be used for the maintenance of the desired temperature over an extended period of time, also after intra-arterial perfusion has been stopped. Optionally, the blanket may be used for heating the rest of the body during an extended period of intra-arterial perfusion with cooling perfusate, intended for maintenance of local brain hypothermia.

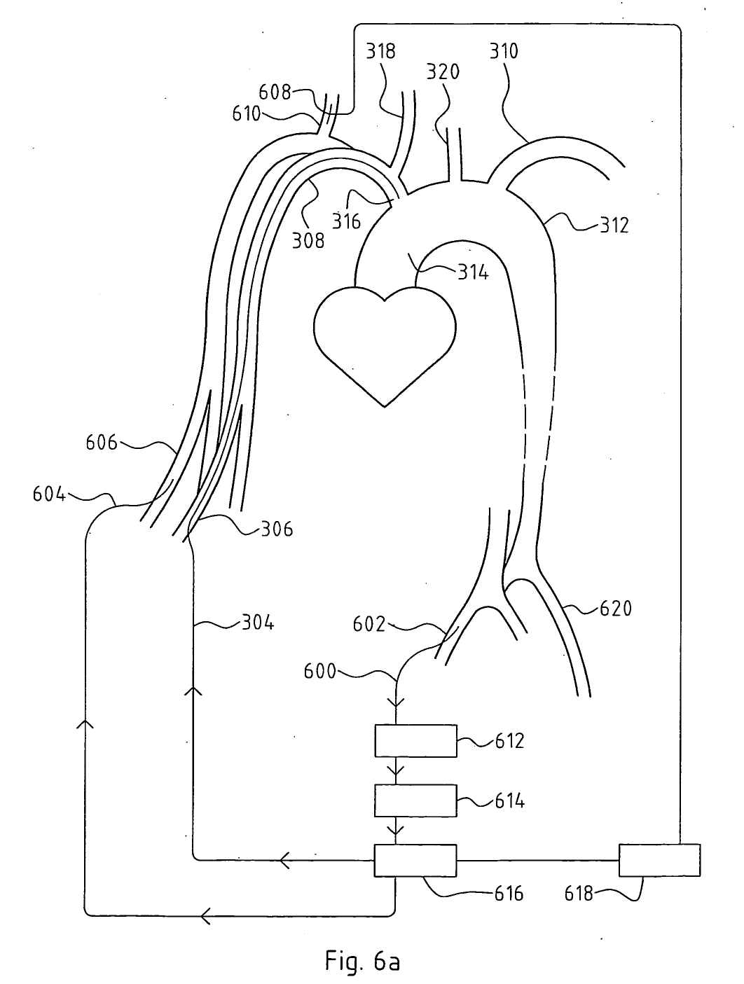

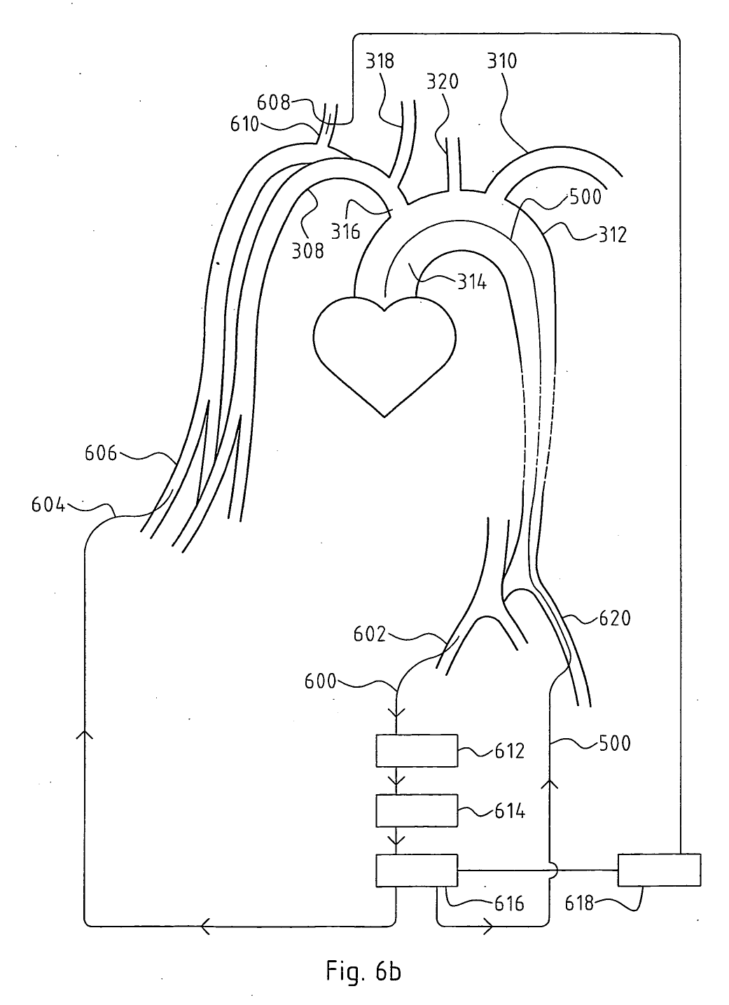

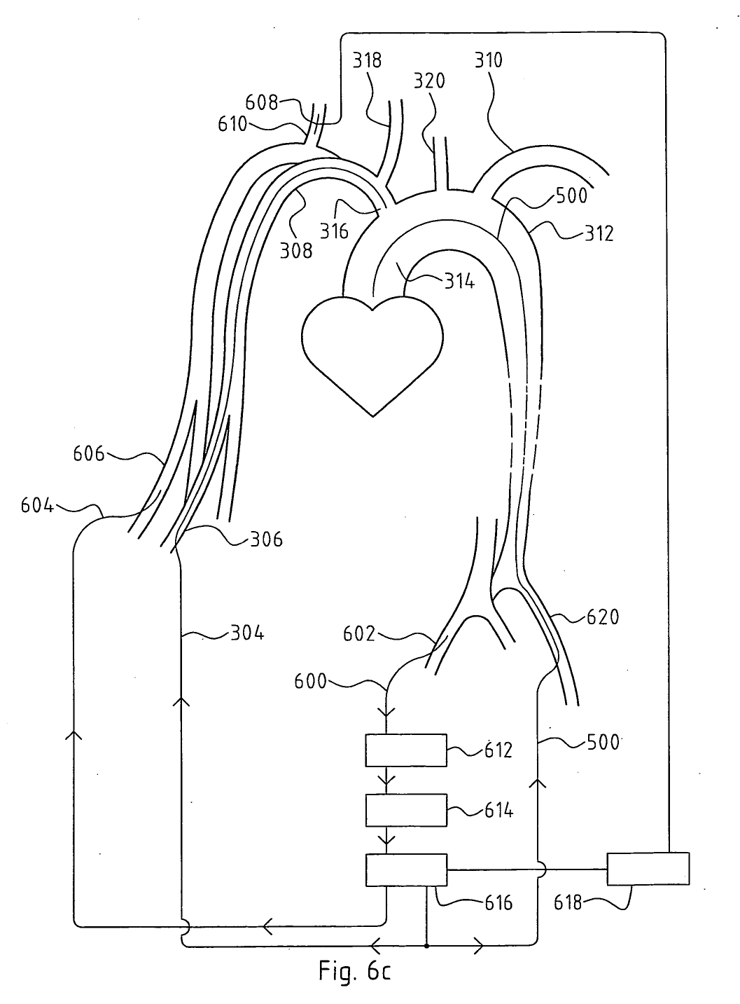

FIG. 6a shows schematically an embodiment of the maintained temperature control phase applied in a human patient. An extraction catheter 600 for extraction of blood is introduced into a central vein or artery of a patient, for example in a femoralis vein 602. Further, a venous catheter 604 is inserted into a vein of the patient, for example the right median vein 606. The extraction catheter 600 is further connected to the venous catheter 604 via a perfusion pump 612, an oxygenator/dexoygenator 614 and a temperature regulator 616, whereby a blood circuit is established supplying heated extracted blood to the patient through the venous catheter 604.

In this embodiment an arterial infusion catheter 304 is introduced into the truncus brachiocephalica 316 through the right radial artery 306 and via the right subclavian artery 308. Further, the arterial infusion catheter is connected to the extraction catheter 600 via the perfusion pump 612, the oxygenator/deoxygenator 614 and the temperature regulator 616, whereby a blood circuit is established supplying cooled extracted blood to the patient via the truncus brachiocephalica 316.

As schematically illustrated in the figure, a temperature sensor 608 is introduced into a jugular vein 610 and possibly connected to a control unit 618 regulating the temperature regulator 616 cooling the extracted blood, whereby the cooling of the brain is controlled or monitored.

In another embodiment of the invention as schematically illustrated in FIG. 6b, an arterial infusion catheter 500 is inserted into the ascending aorta 314 via the left femoral artery 620 and the aortic arch 312. In this embodiment, the cooling blood circuit is established between the extraction catheter 600 and the arterial infusion catheter 500 via the perfusion pump 612, the oxygenator/deoxygenator 614 and the temperature regulator 616. Further, in this embodiment the arterial infusion catheter 500 is the double lumen catheter described with reference to FIG. 5, but it should be understood that the arterial catheter also can be another kind of catheter and alternatively placed in a common carotid artery under the protection of an embolus filter device.

In yet another embodiment of the invention as schematically illustrated in FIG. 6c, three extra-corporeal blood circuits are established. This embodiment of the invention is a combination of the two embodiments previously described with reference to FIGS. 6a and 6b. Firstly, an extra-corporeal blood circuit for heated blood is established between the extraction catheter 600 and the venous infusion catheter 604 via the perfusion apparatus 612, the oxygenator/deoxygenator 614 and the temperature regulating 616. Secondly, an extra-corporeal blood circuit for cooled blood is established between the extraction catheter 600 and the arterial infusion catheter 304 via the perfusion apparatus 612, the oxygenator/deoxygenator 614 and the temperature regulating 616. Thirdly, a third extra-corporeal blood circuit for cooled blood is established between the extraction catheter 600 and the arterial infusion catheter 500 via the perfusion apparatus 612, the oxygenator/deoxygenator 614 and the temperature regulating 616. Thus, in this embodiment having two extra-corporeal blood circuits for cooled blood, a more efficient cooling of the brain is achieved.

Further, in embodiments of the invention the perfusion apparatus 616 is arranged to generate a pulsating flow of infusion solution. This pulsating flow is then synchronized to the heart cycle such that the cooled infusion solution flows up in the left and right carotids, 320 and 318, respectively, when the heart is in systole, i.e. when the heart is contracting and pumping blood to the arteries.

According to another embodiment of the invention, the infusion catheter described above can be positioned in the internal jugular vein on one side of the neck. The infusion catheter is preferably positioned with its tip directed cranially. This can be accomplished by means of puncturing the internal jugular vein with a needle. A guide wire can then be introduced through the needle and into the vein in the cranial direction and an introducer, e.g. an introducer of approximately 9 French, is placed into the vein.

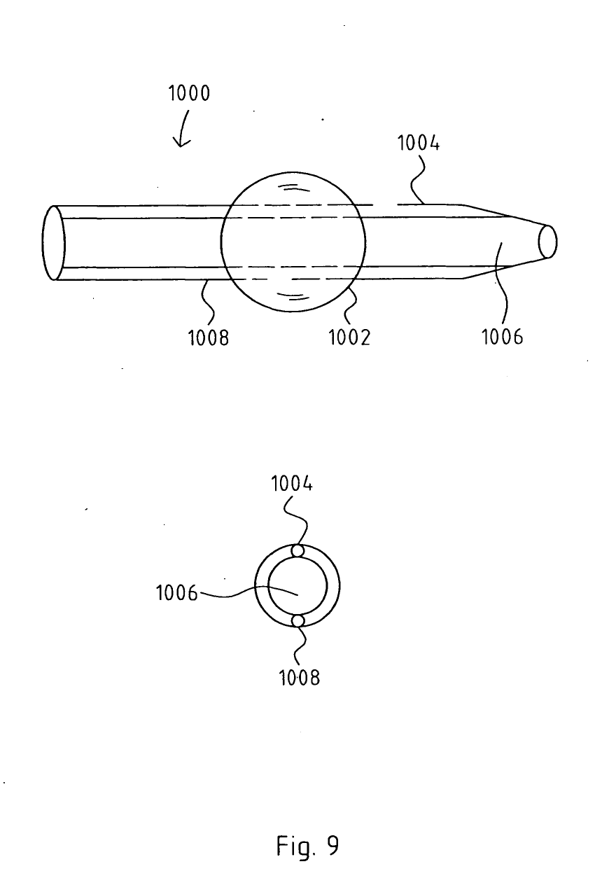

According to an embodiment, the infusion catheter is a triple lumen balloon catheter 1000, cf. FIG. 9, is placed through the introducer and placed with the balloon 1002 at the level of the angle of the mandible. The triple lumen balloon catheter 1000 can for example be an 8 French catheter having a length of approximately 25 centimeters. The three lumens are intended for introduction of a pressure recording means, for infusion of a brain-protective fluid and for inflation/deflation of the balloon.

By means of a pressure recording means inserted via a first lumen 1004 of the catheter 1000, the pressure in the jugular vein can monitored. The first lumen 1004 ends approximately 1.5 cm proximally of the catheter tip.

A Y-connector can be attached to preferably the stopcock of a second lumen 1006 of the catheter 1000. Further a temperature sensor, e.g. a thin wire with a thermistor, can be placed through one of the arms of the Y-connector and through the second lumen 1006 and placed approximately 3 centimeters distally of the tip of the catheter 1000 in order to measure the temperature of the infused fluid. The measured temperature can further be an indication of the presence of systemic blood, i.e. that the balloon does not seal off the vein, since this systemic blood will heat a cooled infusion fluid. Alternatively the thermistor may be preimbedded into the material composing the catheter 1000.

Cold cristalloid solution is infused through a second lumen 1006 of the catheter 1000 with its balloon 1002 inflated, which balloon 1002 is inflated and deflated by means of a third lumen 1008. The infusion rate is regulated in order not to have the vein pressure to exceed about 25 mmHg. The infusion of cristalloid solution is subsequently followed by the infusion of blood extracted from the remaining jugular veins or the femoral veins or arteries. The blood has passed through a cooler, possibly a pump and possibly an oxygenator/deoxygenator passed to the catheter in the jugular vein.

According to this embodiment a double lumen catheter, e.g. an approximately 16 French double lumen catheter, is placed through one of the femoral veins into the inferior vena cava. A first lumen ends approximately 25 mm from the tip of the catheter. A thermistor at the opening of the first lumen is recording the temperature of the blood in the distal part of the inferior vena cava. Blood is then aspirated from the first lumen with a pump, passed through a heater and infused through the tip of the infusion catheter via a second lumen of the infusion catheter. Temperature is in this way automatically regulated in the inferior vena cava to stay between approximately 37 and 36 degrees Celsius. However, blood can also extracted from a femoral artery and the blood flow from the femoral artery can be controlled by a flow controller (not shown) arranged at the catheter in the internal vena jugularis causing the blood to flow due to the pressure difference in the artery and the vein, respectively. Thus, in such an embodiment the pump is not necessary.

Further, as an alternative to the double lumen catheter in the femoral vein, a double lumen catheter can be introduced into the superior vena cava via the other internal vena jugularis.

The Temperature Regulating Device

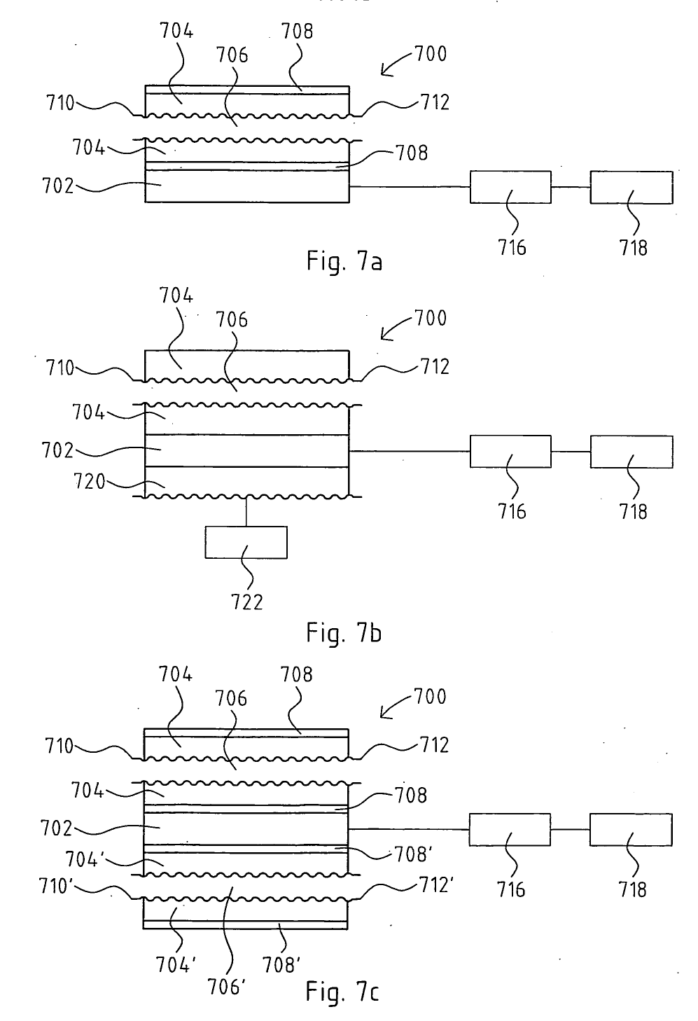

In one embodiment of the invention as schematically illustrated in FIG. 7a, the temperature regulating device or temperature regulator 700 comprises a peltier element 702 and a heat exchanger such as a tube or a conduit 704 of for example folded, twisted or wrinkled stainless steel or another material able to transfer thermal energy. The conduit 704 confines a passage 706 for fluid to be temperature regulated. Thus the conduit 704 has an inlet 710 for fluid to be temperature regulated and an outlet 712 for temperature regulated fluid. If the fluid is to be cooled the conduit 704 should be arranged on the cool side of the peltier element 702 and if the fluid is to be heated the conduit 704 should be arranged on the hot side of the peltier element 702.

In another embodiment of the invention as schematically illustrated in FIG. 7b, a cooling flange 720 is further arranged at the hot side of the peltier element 702, which cooling flange 720 is connected to a fall or another cooling device, e.g. a ventilator 722, whereby the hot side of the peltier element 702 is cooled.

In another embodiment of the invention as schematically illustrated in FIG. 7c, the temperature regulating device 700 is used for both cooling and heating a fluid. In this embodiment, the temperature regulating device 700 is designed as a double heat exchanger comprising a peltier element 702 and two conduits 704,704′ confining passages 706,706′ having inlets 710,710′ and outlets 712,712′. In this embodiment, fluid passing though the passages 706,706′ is cooled and heated, respectively.

Further, the temperature regulating device 700 is provided with a power supply 716 supplying power to the peltier element and a control unit 718 controlling the temperature regulation.

In one embodiment of the invention, the conduit 704 or the conduits 704,704′ is/are provided with a plastic or plastic-like housing or cover 708 or covers 708, 708′, by means of which the conduit 704 or conduits 704,704′ is/are arranged to be attachable and detachable at the peltier element 702. In this embodiment the conduit 704,704′ and the cover 708,708′ is preferably manufactured as a disposable or disposables, preferably as a sterile disposable or disposables provided with for example an anticoagulant agent on the inner surface of the conduit 704,704′.

However, in another embodiment of the invention, the temperature regulating device 700 is manufactured as a disposable and preferably as a disposable having sterile inner surfaces of the conduit 704,704′ not to contaminate the fluid to be temperature regulated and inner surfaces provided with an anticoagulant agent.

The present invention has been described above with reference to exemplifying embodiments, and it is obvious to a person skilled in the art that the invention may be modified in other ways within the scope of the appended claims.

1-77. (canceled)

78. A method for brain hypothermia, comprising the steps of:
inserting into a blood vessel an extraction catheter for extraction of blood;
inserting a first infusion catheter into an internal jugular vein, said first infusion catheter being placed with its tip directed cranially;
establishing a first extra-corporeal blood circuit for cooled blood between said extraction catheter and said first infusion catheter via a pumping means and a temperature regulating device capable of cooling extracted blood;
extracting blood from said blood vessel via said extraction catheter;
cooling said extracted blood by means of said temperature regulating device;
infusing said cooled extracted blood to the brain via said first infusion catheter; and
maintaining cooling and circulation of blood in said first extra-corporeal circuit for a selected period of time.

79. The method of claim 78, further comprising the step of placing a balloon of said first infusion catheter at the level of the angle of the mandible.

80. The method of claim 78, further comprising the steps of introducing a pressure recording means via a first lumen of said first infusion catheter means; and monitoring the pressure in the jugular vein by means of said pressure recording means.

81. The method of claim 80, further comprising the step of regulating the infusion rate in order to provide a vein pressure that is lower than about 25 mmHg.

82. The method of claim 78, further comprising the step of deflating and inflating said balloon by means a third lumen of said first infusion catheter.

83. The method of claim 78, wherein said extraction catheter is placed through one of the femoral veins into the inferior vena cava.

84. The method of claim 83, further comprising the step of placing a thermistor at the end opening of a first lumen of said extraction catheter and by means of said thermistor recording the temperature of the blood in the distal part of the inferior vena cava.

85. The method of claim 78, further comprising the steps of:
inserting a double lumen catheter into a vein of the venous system;
establishing a second extra-corporeal blood circuit between a first lumen of said double lumen catheter and a second lumen of said double lumen catheter via said pumping means and said temperature regulating device capable of heating extracted blood;
leading an amount of said extracted blood from said blood vessel via said first lumen of said double lumen catheter into said second extra-corporeal blood circuit;
heating said amount of extracted blood;
infusing said heated amount of extracted blood to said venous system via said second lumen of said double lumen catheter; and
maintaining heating and circulation in said second extra-corporeal blood circuit for a selected period of time.

86. The method of claim 78, further comprising the step of automatically regulating the temperature of the infused blood in dependence of the brain temperature or the body temperature.

87. The method of claim 85, further comprising the step of automatically regulating the temperature of the infused blood in dependence of the brain temperature or the body temperature.

88. The method of claim 78, further comprising the step of providing a heating blanket configured to keep the body temperature at a desired temperature during brain hypothermia.

89. An infusion catheter for use in a method for brain hypothermia according to claim 78, said infusion catheter being configured for insertion into an internal jugular vein with its tip directed cranially and comprising a first lumen ending approximately 1.5 cm proximally of the tip of the infusion catheter and configured to be connected to means for recording the pressure in said vein, a second lumen for infusion of an infusion solution, and a third lumen connected to a balloon and configured to inflate and deflate said balloon.

90. The infusion catheter of claim 89, wherein said balloon is arranged proximally of said end opening of said first lumen.

91. An equipment for brain hypothermia, comprising:
an extraction catheter being configured to be inserted into a blood vessel for extraction of blood;
a first infusion catheter according to claim 89, said first infusion catheter being configured for insertion into an internal jugular vein and to be placed in said internal jugular vein with its tip directed cranially;
coupling means for establishing an first extra-corporeal blood circuit for cooled blood between said extraction catheter and said first infusion catheter via a pumping means and a temperature regulating device capable of cooling extracted blood; and said equipment being configured to:
extract blood from said blood vessel via said extraction catheter leading an amount of said extracted blood into said first extra-corporeal blood circuit;
cool said amount of said extracted blood;
infuse said cooled extracted blood to the brain via said first infusion catheter; and
maintain cooling and circulation in said first extra-corporeal blood circuit for a selected period of time.

92. An equipment for brain hypothermia, comprising:
an extraction catheter being configured to be inserted into a blood vessel for extraction of blood;
a first infusion catheter according to claim 90, said first infusion catheter being configured for insertion into an internal jugular vein and to be placed in said internal jugular vein with its tip directed cranially;
coupling means for establishing an first extra-corporeal blood circuit for cooled blood between said extraction catheter and said first infusion catheter via a pumping means and a temperature regulating device capable of cooling extracted blood; and said equipment being configured to:
extract blood from said blood vessel via said extraction catheter leading an amount of said extracted blood into said first extra-corporeal blood circuit;
cool said amount of said extracted blood;
infuse said cooled extracted blood to the brain via said first infusion catheter; and
maintain cooling and circulation in said first extra-corporeal blood circuit for a selected period of time.

93. The equipment of claim 91, further comprising:
a double lumen catheter being configured to be inserted into a vein of the venous system; and said equipment further being configured to:
establish a second extra-corporeal blood circuit for heated blood between a first lumen of said double lumen catheter and a second lumen of said double lumen catheter via said pumping means and said temperature regulating device capable of heating extracted blood;
lead an amount of said extracted blood from said blood vessel via said first lumen of said double lumen catheter into said second extra-corporeal blood circuit;
heat said amount of said extracted blood;
infuse said heated amount of extracted blood to said venous system via said second lumen of said double lumen catheter; and
maintain heating and circulation in said second extra-corporeal blood circuit for a selected period of time.

94. The equipment of claim 92, further comprising:
a double lumen catheter being configured to be inserted into a vein of the venous system; and said equipment further being configured to:
establish a second extra-corporeal blood circuit for heated blood between a first lumen of said double lumen catheter and a second lumen of said double lumen catheter via said pumping means and said temperature regulating device capable of heating extracted blood;
lead an amount of said extracted blood from said blood vessel via said first lumen of said double lumen catheter into said second extra-corporeal blood circuit;
heat said amount of said extracted blood;
infuse said heated amount of extracted blood to said venous system via said second lumen of said double lumen catheter; and
maintain heating and circulation in said second extra-corporeal blood circuit for a selected period of time.

95. The equipment of claim 91, further comprising means for automatically regulating the temperature of the infused blood in dependence of the brain temperature or the body temperature.

96. The equipment of claim 93, further comprising means for automatically regulating the temperature of the infused blood in dependence of the brain temperature or the body temperature.

97. The equipment of claim 91, further comprising a heating blanket configured to keep the body temperature at a desired temperature during brain hypothermia.
